(19) United States
(12) Patent Application Publication
(10) Pub. No.: US 2018/0043129 A1
(43) Pub. Date: Feb. 15, 2018

(54) Meditation Aid

(71) Applicant: Gene Raymond Phelps, Sanderson, TX (US)

(72) Inventor: Gene Raymond Phelps, Sanderson, TX (US)

(21) Appl. No.: 15/234,100

(22) Filed: Aug. 11, 2016

(51) Int. Cl. A61M 21/02

(52) U.S. Cl. CPC A61M 21/02; A61M 2021/0044

(57) ABSTRACT

A meditation aid is provided. The meditation aid has three ritual objects. A first outer set of six bar members are connected in a pyramid formation. The pyramid formation has four corner portions defining six medial connecting points. A second inner set of twelve bar members are connected in a penta-tetrahedron formation. The penta-tetrahedron formation is centrally suspended within the pyramid formation by a set of four strut members. The four strut members are evenly disposed in connection with each of a respective one of each of the four corner portions. A third innermost set of three medially connected bar members is configured in a jacks-like star formation. The opposite ends, of the star formation bar members, are evenly disposed in spaced relationship for connection to a respective one of each of the six medial connecting points.

### CROSS REFERENCE TO RELATED APPLICATIONS

Not Applicable.

### STATEMENT OF FEDERALLY SPONSORED RESEARCH

Not Applicable.

### BACKGROUND OF THE INVENTION

### Field of the Invention

The present invention relates to meditation aids. In particular, it relates to a meditation aid showing differing configurations when viewed from a variety of different viewpoints.

### Description of the Related Art

Meditation is a form of concentration where an individual induces a mode of consciousness. The object is for the individual to either realize some benefit or for the mind to simply acknowledge its content without becoming identified with that content. Meditation takes the form of a broad variety of practices that includes techniques designed to promote relaxation, build internal energy or life force, and with a goal of developing emotions such as compassion, love, patience, generosity, or forgiveness. A particularly ambitious form of meditation aims at effortlessly sustained single-pointed concentration meant to enable its practitioner to enjoy an indestructible sense of well-being while engaging in any life activity.

Meditation has been practiced since antiquity as a component of numerous religious traditions and beliefs. Meditation often involves an internal effort to self-regulate the mind in some way. Meditation is also used to clear the mind and ease health concerns, such as high blood pressure, depression, and anxiety. Meditation aids, such as Prayer beads or other ritual objects are commonly used during meditation in order to keep track of, or remind, the practitioner about some aspect of that training.

What is needed, however, is a novel meditation aid which is useful to generate an infinite number of observable ritual objects, when observed from an infinite number of viewpoints, in order to remind the practitioner about one aspect of that meditation training, when observed from one viewpoint, but which reminds the practitioner of yet another aspect of the practitioners meditation training when viewed from yet another viewpoint. The present invention satisfies these needs.

### BRIEF SUMMARY OF THE INVENTION

It is therefore an object of the present invention to provide a novel meditation aid which is useful to generate an infinite number of observable ritual objects, when observed from an infinite number of viewpoints, in order to remind the practitioner about one aspect of that meditation training, when observed from one viewpoint, but which reminds the practitioner of yet another aspect of the practitioners meditation training when viewed from yet another viewpoint

To overcome the problems associated with the prior art and in accordance with the purpose of the present invention, as embodied and broadly described herein, briefly, a meditation aid is provided. The meditation aid is three ritual objects. The object maybe used separately or and constructed together as a single built-up member. The first object is a pyramid formation having a first outer set of six bar members. The pyramid formation has four corner portions defining six medial connecting points. A second object has an inner set of bar members being connected in a tetrahedron formation. The tetrahedron formation is suspended within the pyramid formation by at least one strut member. The third object is an innermost set of three medially connected bar members being configured in a jacks like star formation. The opposite ends, of the star formation, bar members are evenly disposed in spaced relationship for connection to a respective one of each of the six medial connecting points.

Additional advantages of the present invention will be set forth in part in the description that follows and in part will be obvious from that description or can be learned from practice of the invention. The advantages of the invention can be realized and obtained by the method particularly pointed out in the appended claims.

### BRIEF DESCRIPTION OF THE SEVERAL VIEWS OF THE DRAWINGS

The accompanying drawings, which are incorporated in and which constitute a part of the specification, illustrate at least one embodiment of the invention and, together with the description, explain the principles of the invention.

FIG. 1 is left hand perspective view of a preferred embodiment of the present invention showing the three ritual objects assembled together.

FIG. 2 is right hand perspective view of a preferred embodiment of the present invention showing the three ritual objects assembled together.

FIG. 3 is a left hand exploded perspective view showing each of the respective ritual objects in a preferred relative position for construction of the presently preferred embodiment.

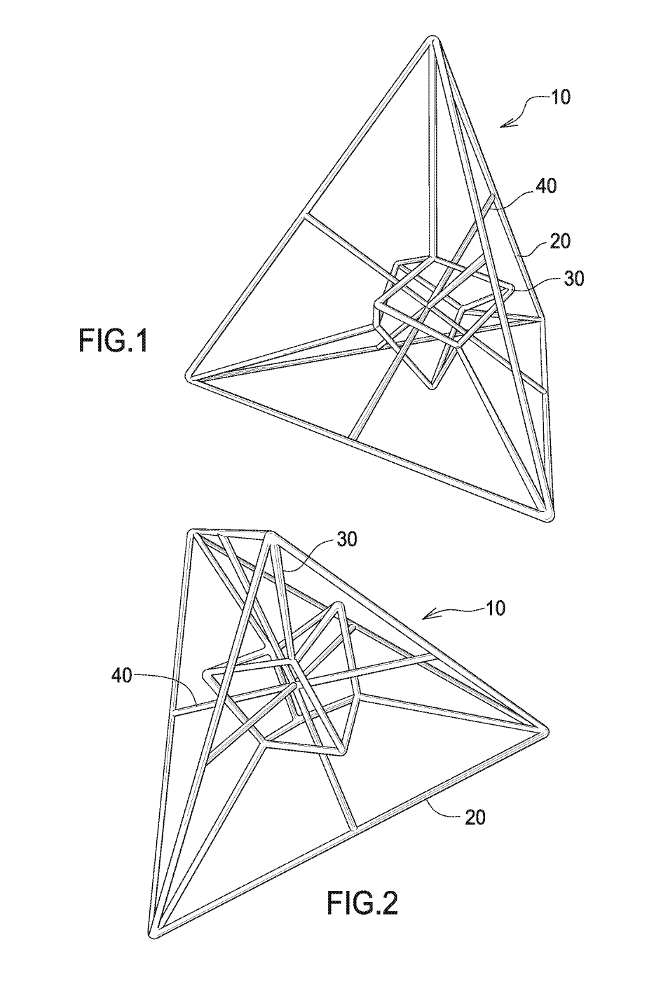

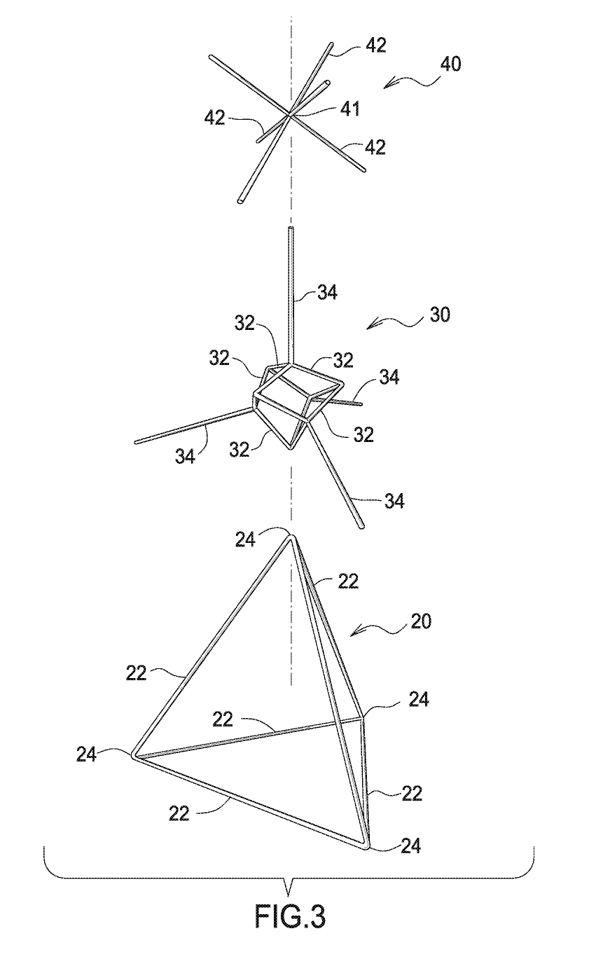

### DETAILED DESCRIPTION OF THE DRAWINGS

Unless specifically defined otherwise, all technical or scientific terms used herein have the same meaning as commonly understood by one of ordinary skill in the art to which this invention belongs.

Although any methods and materials similar or equivalent to those described herein can be used in the practice or testing of the present invention, the preferred methods and materials are now described. Reference now will be made in detail to the presently preferred embodiments of the invention, examples of which are illustrated in the accompanying drawings, wherein like numerals represent like features.

The present invention provides a meditation aid. The meditation aid includes three ritual object which may be a separate set of objects or, with the presently preferred embodiment, constructed together as a single built-up member. With the preferred embodiment, the meditation aid has a first ritual object being an outer pyramid formation, a second object being a central or middle penta-tetrahedron, and a third object being an inner multi-axis star formation. The middle penta-tetrahedron and inner star formations, may be separate, but are desirably suspended within an interior or central portion, of the pyramid.

The meditation aid 10 is desirably constructed as a built-up formation with a plurality of rigid bar members 22, 32, 42 but may be formed as a molded or blown glass construction. The bar members 22, 32, 42 are desirably formed of wood, a brass, copper, aluminum, gold, silver, platinum, or titanium metal wire, but may also be a polymer or blown glass, or any combination thereof. The bar members 22, 32, 42 are preferably a round wire, but may also be four sided, or elliptical in cross-section. Thus, for example, the pyramid formation 20 may be formed of a polished metal wire, with the middle penta-tetrahedron formation 30 being formed of a blown glass assembly and being adhered to the pyramid formation 20 bar members 22 using an adhesive.

The bar members 22, 32, 42 are desirably connected using a joint construction formed with a solder, weld, adhesive, or a mold process, in any manner which is well known in the art of constructing built-up members. Where it is desirable to build the present invention, or a portion thereof, with a glass construction, the present invention is desirably formed using any blown glass material, such as Pyrex ^{TM}, and any blown glass method which is well known in the art. The bar members 22, 32, 42, for use with the present invention, are desirably stock rod or tubular members having a diameter in range of between 1.59-4.76 millimeters, depending upon the intended material used in the overall construction of the meditation aid 10.

The outer pyramid is desirably constructed to include a first outer set of six bar members 22 being connected in a pyramid formation 20. The pyramid formation 20 has four corner portions 24 defining six medial connecting points. In this manner, a monumental pyramid structure is formed with a triangular base and sloping sides that meet in a point at the top. Rotation of the pyramid enables a wide variety of different view-points.

A second inner set of twelve bar members 32 are desirably connected in a penta-tetrahedron formation 30. The penta-tetrahedron formation 30 is suspended within the pyramid formation 20 by a set of four strut members 34. The four strut members 34 are evenly disposed in connection with each of a respective one of each of the four corner portions 24.

A third innermost set of three medially connected 41 bar members 42 is configured in a jacks-like star formation 40. The opposite ends, of the star formation 40, bar members 42 are evenly disposed in spaced relationship for connection to a respective one of each of the six medial connecting points.

While the present invention has been described in connection with the illustrated embodiments, it will be appreciated and understood that certain modifications may be made without departing from the true spirit and scope of the invention.

I claim:

1. A meditation aid, comprising:
(a) an outer set of six bar members being connected in a pyramid formation, said pyramid formation having four corner portions defining six medial connecting points.

2. The meditation aid according to claim 1, further comprising a second inner set of bar members being connected in at least one tetrahedron formation, said tetrahedron formation being suspended within said pyramid formation by at least one strut member.

3. The meditation aid according to claim 1, further comprising a set of three medially connected bar members configured in a star formation, wherein each of said three bar members includes opposite ends thereof, and wherein each of said opposite ends being evenly disposed in a spaced relationship for connection to a respective one of each of said six connecting points at said corner portions.

4. The meditation aid according to claim 1, wherein said bar members are a metal wire.

5. The meditation aid according to claim 1, wherein said bar members are a polymer rod.

6. The meditation aid according to claim 1, wherein said bar members are a glass rod.

7. The meditation aid according to claim 1, wherein said bar members are wood.

8. The mediation aid according to claims 1, wherein said bar members are in a diameter range of 1.59-4.76 millimeters.

9. The meditation aid according to claim 2, wherein said at least one tetrahedron formation is a penta-tetrahedron construction being suspended within said pyramid formation by a set of four strut members, wherein said four strut members being evenly disposed in connection with a respective one of each of said four corner portions.

10. The meditation aid according to claim 4, wherein said wire is selected from a group consisting of brass, copper, aluminum, titanium, gold, silver, platinum, and carbon steel.

11. A meditation aid, comprising:
(a) a first outer set of six bar members being connected in a pyramid formation, said pyramid formation having four corner portions defining six medial connecting points;
(b) a second inner set of twelve bar members being connected in a penta-tetrahedron formation, said penta-tetrahedron formation being suspended within said tetrahedral pyramid formation by a set of four strut members, wherein each of said four strut members being evenly disposed in connection with a respective one of each of said four corner portions; and
(c) a third innermost set of three medially connected bar members configured in a star formation, wherein each of said three bar members includes opposite ends thereof, and wherein each of said opposite ends being evenly disposed in an evenly spaced relationship for connection to a respective one of each of said six connecting points at said corner portions.

12. The meditation aid according to claim 11, wherein said toy comprises twenty-eight straight bar members.

13. The meditation aid according to claim 11, wherein said toy includes nineteen connection points.

14. The meditation aid according to claim 11, wherein said bar members are a wire.

15. The meditation aid according to claim 11, wherein said bar members are a polymer.

16. The meditation aid according to claim 11, wherein said bar members are a glass rod.

17. The mediation aid according to claim 11, wherein said bar members are in a diameter range of 1.59-4.76 millimeters.

18. The meditation aid according to claim 11, wherein said bar members are wood.

19. The meditation aid according to claim 14, wherein said wire is selected from a group consisting of brass, copper, aluminum, titanium, gold, silver, platinum, and carbon steel.
